(19) United States
(12) Patent Application Publication
(10) Pub. No.: US 2026/0345740 A1
(43) Pub. Date: Nov. 5, 2026

(54) CRYSTALLINE PROTEIN COMPOSITIONS AND METHODS FOR IMPROVING STABILITY, DISPERSIBILITY AND SHELFLIFE OF NUTRITIONAL PRODUCTS

(71) Applicant: Glanbia Performance Nutrition Limited, Downers Grove, IL (US)

(72) Inventor: Kenneth Czaplewski, Westmont, IL (US)

(21) Appl. No.: 19/645,934

(22) Filed: Apr. 13, 2026

Related U.S. Application Data
(60) Provisional application No. 63/791,117, filed on Apr. 18, 2025.

(51) Int. Cl. A61K 38/39; A23J 3/08; A61K 31/07; A61K 31/122; A61K 31/197; A61K 31/355; A61K 31/375; A61K 31/4188; A61K 31/4415; A61K 31/455; A61K 31/51; A61K 31/519; A61K 31/525; A61K 31/593; A61K 31/714; A61K 33/02; A61K 33/04; A61K 33/06; A61K 33/18; A61K 33/22; A61K 33/24; A61K 33/26; A61K 33/30; A61K 33/34; A61K 35/20; A61K 38/17

(52) U.S. Cl. CPC A61K 38/39; A23J 3/08; A61K 31/07; A61K 31/122; A61K 31/197; A61K 31/355; A61K 31/375; A61K 31/4188; A61K 31/4415; A61K 31/455; A61K 31/51; A61K 31/519; A61K 31/525; A61K 31/593; A61K 31/714; A61K 33/02; A61K 33/04; A61K 33/06; A61K 33/18; A61K 33/22; A61K 33/24; A61K 33/26; A61K 33/30; A61K 33/34; A61K 35/20; A61K 38/1722

(57) ABSTRACT

The present disclosure relates to methods of crystallizing proteins for use in nutritional compositions, to nutritional compositions using crystallized whey and other proteins for nutritional, nutraceutical, beverage, and supplement compositions having improved shelf life and stability, and to methods to use crystalline whey proteins to increase crystal density, reduce hygroscopicity, and improve dispersibility of crystalline proteins in consumer nutritional, nutraceutical, food, beverage, and supplement products.

### CROSS-REFERENCE TO RELATED APPLICATIONS

The present application claims priority to U.S. provisional application Ser. No. 63/791,117, filed Apr. 18, 2025, herein incorporated by reference in its entirety.

### TECHNICAL FIELD

This invention relates to nutritional compositions using crystallized whey and other proteins for nutritional, nutraceutical, beverage, and supplement compositions, and methods to use crystalline whey proteins to increase crystal density, reduce hygroscopicity, and improve dispersibility of crystalline proteins in consumer nutritional, nutraceutical, food, beverage, and supplement products.

### BACKGROUND

Protein powders are notoriously difficult to disperse in water. For instance, whey protein powder when added directly to water remains on the surface of the water and does not readily disperse unless vigorous agitation, shaking, or stirring is applied to the mixture. This is a common inconvenience experienced by consumers of whey protein health and other supplements to produce whey protein supplement beverages.

Not only is the low dispersibility of protein powders in water an undesirable property, but also paradoxically the high level of hygroscopicity that is characteristic of protein powders, particularly whey protein powder, can cause reduced shelf life of whey protein powders and products incorporating whey proteins in their formulations. This increase in water absorption leads to degradation, oxidation, caking/clumping, and other processes that reduce the organoleptic properties of consumer products incorporating protein powders, particularly whey protein powder, thereby shortening the shelf-life of those products.

Given the popularity of health nutritional, nutraceutical, beverage and food products incorporating protein powders particularly whey protein powder, in their formulations, there is an ongoing need for compositions and formulations that avoid the reduced shelf life and improve the dispersibility properties of protein powders.

Thus, there exists a need for compositions and formulations containing crystalline proteins to meet these and other needs. The compositions, formulations, and methods of the present invention meet these and other needs as detailed below.

### SUMMARY

In one embodiment a dietary supplement, food product, or nutraceutical composition is described for use in supporting health in a human subject, comprising a blend of crystalline protein in a powder formulation with improved shelf life. The nutritional compositions, formulations, and supplements described herein may be formulated for oral administration as a nutritional, nutraceutical, food, beverage, or supplement product.

In one embodiment a dietary supplement, food product, or nutraceutical composition is described for use in supporting health in a human subject, comprising a blend of crystalline whey protein powder and other nutritional, nutraceutical, beverage, and food compositions in a powder formulation with improved shelf life. The nutritional compositions, formulations, and supplements described herein may be formulated for oral administration as a nutritional, nutraceutical, food, beverage, or supplement product.

In one embodiment a method of producing a crystalline protein is described for use in a dietary supplement, food product, or nutraceutical formulation for use in supporting health in a human subject, comprising crystallization of a protein resulting in a protein crystal with reduced hygroscopicity thus improved shelf life, and low deliquescence properties. The formulations described herein may be formulated for oral administration as a nutritional, nutraceutical, food, beverage, or supplement product.

In one embodiment a method of producing a crystalline whey protein is described for use in a dietary supplement, food product, or nutraceutical formulation for use in supporting health in a human subject, comprising crystallizing a protein resulting in a protein crystal with reduced hygroscopicity thus improved shelf life, and low deliquescence properties. The formulations described herein may be formulated for oral administration as a nutritional, nutraceutical, food, beverage, or supplement product.

In one embodiment a method is described for improving shelf life and stability of a protein powder component in a formulation comprising crystallizing the protein into crystalline form of the protein having reduced hygroscopicity, higher density and dispersibility, and improved solubility properties. The formulations described herein may be formulated for oral administration as a nutritional, nutraceutical, food, beverage, or supplement product.

In one embodiment a method is described for improving shelf life and stability of a whey protein powder component in a dietary supplement, food, beverage, or nutraceutical formulation, comprising crystallizing whey protein into crystalline form of the protein having reduced hygroscopicity, higher density and dispersibility, and improved solubility properties.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 depicts crystalline protein of Isowater™ and β-lactoglobulin proteins having reduced hygroscopicity than their amorphous counterparts.

FIG. 2 depicts density analysis of amorphous and crystalline Isowater™ and β-lactoglobulin proteins having higher densities than amorphous protein.

FIG. 3 depicts polarized light microscopy of crystalline protein particles demonstrating birefringent properties consistent with crystalline state.

### DETAILED DESCRIPTION

The present disclosure encompasses embodiments of a dietary supplement, food product, or nutraceutical composition for use in supporting health in a human subject, comprising one or more crystalline proteins in a powder formulation with improved shelf life. The nutritional compositions, formulations, and supplements described herein may be formulated for oral administration as a nutritional, nutraceutical, food, beverage, or supplement product.

In another embodiment, the compositions encompass a dietary supplement, food product, or nutraceutical composition for use in supporting health in a human subject, comprising a blend of crystalline whey protein powder and other nutritional, nutraceutical, beverage, and food compositions in a powder formulation with improved shelf life. The nutritional compositions, formulations, and supplements described herein may be formulated for oral administration as a nutritional, nutraceutical, food, beverage, or supplement product.

In another embodiment, the methods of the invention comprise a method of producing a crystalline protein for use in a dietary supplement, food product, or nutraceutical formulation is described for use in supporting health in a human subject, comprising crystallizing a protein resulting in a protein crystal with reduced hygroscopicity thus improved shelf life, and low deliquescence properties. The supplements described herein may be formulated for oral administration as a nutritional, nutraceutical, food, beverage, or supplement product.

In another embodiment, the methods of the invention comprise a method of producing a crystalline whey protein for use in a dietary supplement, food product, or nutraceutical formulation is described for use in supporting health in a human subject, comprising crystallizing whey protein resulting in a protein crystal with reduced hygroscopicity thus improved shelf life, and low deliquescence properties. The supplements described herein may be formulated for oral administration as a nutritional, nutraceutical, food, beverage, or supplement product.

In another embodiment, the methods of the invention comprise a method for improving shelf life and stability of a protein powder component in a dietary supplement, food, beverage, or nutraceutical formulation, comprising crystallizing the protein into crystalline form of the protein having reduced hygroscopicity, higher density and dispersibility, and improved solubility properties. The supplements described herein may be formulated for oral administration as a nutritional, nutraceutical, food, beverage, or supplement product.

In another embodiment, the methods of the invention comprise a method for improving shelf life and stability of a whey protein powder component in a dietary supplement, food, beverage, or nutraceutical formulation, comprising crystallizing whey protein into crystalline form of the protein having reduced hygroscopicity, higher density and dispersibility, and improved solubility properties. The supplements described herein may be formulated for oral administration as a nutritional, nutraceutical, food, beverage, or supplement product.

In another embodiment, the compositions comprise macronutrients such as carbohydrates, sugars, starches, fiber, fats, oils, saturated fats, monounsaturated fatty acids, polyunsaturated fatty acids, essential fatty acids, and proteins used in nutritional, nutraceutical, food, beverage, or supplement products. Same as list above.

In another embodiment, the compositions comprise micronutrients such as vitamin supplementation, minerals, and other micronutrients used in nutritional, nutraceutical, food, beverage, or supplement products for example choline, inositol, flavonoids, bioflavonoid, and carnitine Additional micronutrients used with the crystallized protein in consumer formulations include Creatine, betaine, prebiotics, probiotics, postbiotics, melatonin, biotin, ashwaganda, astragalus, amino acids, branched chain amino acids, caffeine, carnitine, chamomile, huperzine, lycopene, nootropics, yohimbe inulin.

In another embodiment, the compositions comprise vitamins such as vitamin A, vitamins B1, B2, B3, B5, B6, B7, B9 and B12, vitamin C, vitamin D and D3, vitamin E, vitamin K, used in nutritional, nutraceutical, food, beverage, or supplement products.

In another embodiment, the compositions comprise minerals such as boron, calcium, chromium, copper, iron, iodine, magnesium, molybdenum, potassium, selenium, zinc, and chloride used in nutritional, nutraceutical, food, beverage, or supplement products. Minerals used with the crystallized protein in consumer formulations include but are not limited to: Sodium, magnesium, potassium, calcium, phosphorus, sulfur, chlorine, Molybdenum, manganese, iron, cobalt, copper, zinc, selenium, bromine, iodine, Lithium, boron, fluorine, silicon, vanadium, nickel.

In another embodiment, the present disclosure encompasses embodiments of a composition that comprises nutrients and supplements used in health supplements, for example amino acid supplements, creatine, choline, betaine, melatonin, according to one embodiment.

In another embodiment, the protein compositions are derived from nutritional protein powders commonly used in nutraceutical, food, beverage, or supplement products. Proteins used with the crystallized protein in consumer formulations include milk, casein, whey, beta lactoglobulin, alpha lactalbumin, immunoglobulin, bovine serum albumin, lactoferrin, soy, pea, hempseed, quinoa, nuts, algae, rice, mung bean, chia, kale, bean, grasses (wheat, barley, oat), seeds (pumpkin, sunflower).

In another embodiment, the whey protein compositions are derived from animal milks for example but not limited to cow, goat, and sheep milk, among others common only used for human consumption. Alternative animal milks used with the crystallized protein in consumer formulations include buffalo, goat, and the like.

In another embodiment, the additional components in the nutraceutical, nutritional, food, beverage or supplement compositions are derived from any one of or any combination of proteins, vitamins, micronutrients, minerals, and other supplements used to promote health in a human subject.

As used herein, an “effective amount” or an “amount effective for” is defined as an amount effective, at dosages and for periods of time necessary, to achieve a desired biological result, such as reducing, preventing, or treating a disease or condition and/or inducing a particular beneficial effect. The effective amount of compositions of the disclosure may vary according to factors such as age, sex, and weight of the individual. Dosage regime may be adjusted to provide the optimum response. Several divided doses may be administered daily, or the dose may be proportionally reduced as indicated by the exigencies of an individual's situation. As will be readily appreciated, a composition in accordance with the present disclosure may be administered in a single serving or in multiple servings spaced throughout the day. As will be understood by those skilled in the art, servings need not be limited to daily administration, and may be on an every second or third day or other convenient effective basis. The administration on a given day may be in a single serving or in multiple servings spaced throughout the day depending on the exigencies of the situation.

The method described herein results in crystalline proteins having reduced hygroscopicity, thereby improving shelf life and stability of the crystalline protein, and improved long term organoleptic properties in nutritional, nutraceutical, food, beverage, and supplement products.

In certain embodiments, the compositions comprising one or more proteins can include one or more dry carriers selected from the group consisting of trehalose, maltodextrin, rice flour, microcrystalline cellulose, magnesium stearate, inositol, fructooligosaccharide, galactooligosaccharide, dextrose, and the like. In certain embodiments, the dry carrier can be added to the compositions comprising one or more of the above bacterial strains in a weight percentage of from about 1% to about 95% by weight of the composition.

In certain embodiments, the compositions comprising one or more of the above components can include one or more liquid or gel-based carriers, selected from the group consisting of water and physiological salt solutions, urea, alcohols and derivatives thereof (e.g., methanol, ethanol, propanol, butanol), glycols (e.g., ethylene glycol, propylene glycol), and the like; natural or synthetic flavorings and food-quality coloring agents, all compatible with the organism; thickening agents selected from the group consisting of corn starch, guar gum, xanthan gum, and the like; one or more spore germination inhibitors selected from the group consisting of hyper-saline carriers, methylparaben, guar gum, polysorbate, preservatives, and the like. In certain embodiments, the one or more liquid or gel-based carrier(s) can be added to the compositions comprising one or more of the above bacterial strains in a weight/volume percentage of from about 0.6% to about 95% weight/volume of the composition. In certain embodiments, the natural or synthetic flavoring(s) can be added to the compositions comprising one or more of the above bacterial strains in a weight/volume percentage of from about 3.0% to about 10.0% weight/volume of the composition. In certain embodiments, the coloring agent(s) can be added to the compositions comprising one or more of the above bacterial strains in a weight/volume percentage of from about 1.0% to about 10.0% weight/volume of the composition. In certain embodiments, the thickening agent(s) can be added to the compositions comprising one or more of the above bacterial strains in a weight/volume percentage of about 2% weight/volume of the composition.

### Delivery System

Suitable dosage forms include tablets, capsules, solutions, suspensions, powders, gums, and confectionaries. Sublingual delivery systems include, but are not limited to, dissolvable tabs under and on the tongue, liquid drops, and beverages. Edible films, hydrophilic polymers, oral dissolvable films, or oral dissolvable strips can be used. Candy (rock candy, pop rocks, pixie sticks, stick candy, cubes for drinks, ring pops, sprinkles, toppings, stick packs, keto applications, bar texture modifier, throat lozenge, seasoning (alone or in combination with salt, herbs), inclusion, encapsulant, fill agent).

For oral administration, crystallized proteins may be further combined with one or more solid inactive ingredients for the preparation of tablets, capsules, pills, powders, granules, or other suitable dosage forms. For example, an active agent may be combined with at least one excipient selected from the group consisting of fillers, binders, humectants, disintegrating agents, solution retarders, absorption accelerators, wetting agents, absorbents, and lubricating agents. Other useful excipients include, but are not limited to, magnesium stearate, calcium stearate, mannitol, xylitol, sweeteners, starch, carboxymethylcellulose, microcrystalline cellulose, silica, gelatin, silicon dioxide, and the like.

The components of the compositions administered according to the methods of the present disclosure can be administered in a wide variety of oral dosage forms, and consumer products. It will be obvious to those skilled in the art that the following dosage forms may comprise, in certain embodiments, in addition to the crystallized proteins, an active component, either a chemical compound of the present disclosure or an acceptable salt of a chemical compound of the present disclosure.

For preparing nutraceutical compositions to be administered according to the methods of the present disclosure, nutraceutically acceptable carriers can be either solid or liquid. Solid form preparations include powders, tablets, pills, capsules, and cachets. A solid carrier can be one or more substances that may also act as diluents, flavoring agents, solubilizers, lubricants, suspending agents, binders, preservatives, tablet disintegrating agents, or encapsulating materials.

In powders, the carrier is a finely divided solid, which is in a mixture with the finely divided active component. In tablets and capsules, the active component is mixed with the carrier having the necessary binding capacity in suitable proportions and compacted in the shape and size desired.

In certain embodiments, powders and tablets and capsules administered according to methods of the present disclosure preferably may contain from five or ten to about seventy percent of the active compounds. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without additional carriers, is surrounded by a carrier, which is thus in association with it. Similarly, tablets, powders, capsules, pills, sachets, and lozenges are included. Tablets, powders, capsules, pills, sachets, and lozenges can be used as solid forms suitable for oral administration.

Liquid preparations include, but are not limited to, solutions, suspensions, and emulsions, for example, water or water-propylene glycol solutions. The compositions may take such forms as suspensions, solutions, or emulsions in oily or aqueous vehicles, and may contain formulation agents such as suspending, stabilizing, and/or dispersing agents. Oil carriers include, but are not limited to, sunflower oil, olive oil, corn oil, soybean oil, cranberry seed oil, algal oil, palm oil, coconut oil, and rice bran oil. Alternatively, the active ingredient may be in powder form, obtained by aseptic isolation of sterile solid or by lyophilization from solution, for constitution with a suitable vehicle, e.g., sterile, pyrogen-free water, before use.

Aqueous solutions suitable for oral use can be prepared by dissolving the crystallized proteins and active components in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well-known suspending agents, for example to improve organoleptic properties.

Compositions suitable for topical administration in the mouth, or buccal, or sublingual administration include, but are not limited to: lozenges comprising the active agent in a flavored base, usually sucrose and acacia or tragacanth; pastilles comprising the active ingredient in an inert base such as gelatin and glycerin or sucrose and acacia; and mouthwashes comprising the active ingredient in suitable liquid carrier.

The nutraceutical preparations are preferably in unit dosage forms. In such form, the preparation is subdivided into unit doses containing appropriate quantities of the active components. The unit dosage form can be a packaged preparation, the package containing discrete quantities of preparation, such as packaged powders, tablets, capsules, and powders in vials or ampoules. Also, the unit dosage form can be a capsule, tablet, sachet, or lozenge itself; or it can be the appropriate number of any of these in packaged form.

Powders, tablets, capsules, and lozenges for oral administration and liquids for oral use are preferred dosage form compositions.

Further details on techniques for formulation and administration may be found in the latest edition of REMINGTON'S PHARMACEUTICAL SCIENCES (Mack Publishing Co., Easton, PA).

### Routes of Administration

The compositions and compounds may be administered by any route, including, but not limited to, oral, sublingual, buccal, or as an oral spray.

The methods described above may be further understood in connection with the following Examples. In addition, the following non-limiting examples are provided to illustrate the invention. However, the person skilled in the art will appreciate that it may be necessary to vary the procedures for any given embodiment of the invention, e.g., vary the order or steps.

The invention described herein is embodied in the following non-limiting examples.

### Example 1

### Polarized Light Microscopy Analysis of Crystalline Proteins.

### Materials

Arla Isowater™ lot #P090263; Perfect Day Whey β-lactoglobulin lot #22092-CAP are used as protein crystallized and analyzed.

### Methods

Polarized light microscopy (PLM)—images of the protein particles are taken on a Leica DM750 light microscope using a polarized light filter and a 530 nm light filter by mounting the particles on a glass slide and taking images with the 40× objective and a digital camera.

### Isowater™ Protein Crystallization

Various Isowater™ protein crystallization samples are tested to determine protein saturation in solution as detailed in Table 1 below.

TABLE 1

| | | | | | Protein | Conc | Temp | |
|---|---|---|---|---|---|---|---|---|
| Sample | Date | gH2O | Protein | Lot# | (g) | (g/L) | (°C) | Note |
| 1 | Oct. 17, 2024 | 500 | Isowater | P090263 | 30.14 | 60.28 | 37.30 | Not |
| | | | | | | | | saturated. |
| | | | | | | | | Add more |
| | | | | | | | | protein |
| | | | | | | | | next time |
| 2 | Oct. 21, 2024 | 500 | Isowater | P090263 | 100.21 | 200.4 | 42.60 | Still not |
| | | | | | | | | saturated |
| 3 | Oct. 21, 2024 | 250 | Isowater | P090263 | 75.05 | 300.2 | 52.30 | Saturation |
| | | | | | | | | unknown |

Table 1 shows that the first Isowater™ protein solution contains 30 g of protein dissolved in 500 mL of water that was heated to 37 °C. Once the mixing was stopped, the bubbles dissipated and the solution became transparent, suggesting a saturated solution had not been created since no particles were present having all been dissolved. Sample 2 contained 100 g of Isowater™ protein in 500 mL of water that was heated to 43 °C. Again, once the mixing was stopped, the bubble dissipated and the solution became transparent, suggesting the solution was not saturated. The third sample contained 75 g of Isowater™ in 250 mL of water that was heated to 52 °C. It was unclear if this solution was saturated. Higher concentrations were not attempted. Each solution was placed in the hood and the water was allowed to evaporate. After 18 days for sample 1 and 24 days for samples 2 and 3, the beakers were removed from the hood and the crystalline materials were scraped from the bottom of the beakers and collected for evaluation.

β-lactoglobulin Protein Crystallization.

As shown in Table 2, a fourth sample is made containing 50 g of the Perfect Day Whey™ β-lactoglobulin protein in 250 ml of water heated to 53 °C. It was unclear if this solution was saturated. The beaker was placed in the hood for 16 days where the water was allowed to evaporate, and the crystalline materials was scraped from the bottom and collected for analysis.

TABLE 2

| Sample | g H₂O | Protein | Lot # | g Protein | Conc (g/L) | Temp°C | Note |
|---|---|---|---|---|---|---|---|
| 4 | 250 | Perfect | 22092- | 50.10 | 200.4 | 52.80 | Saturation |
| | | Day ™ | CAP | | | | unknown |
| | | Whey | | | | | |

### Results

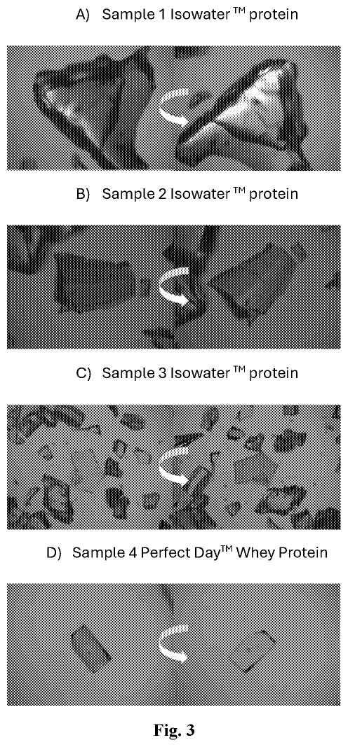

Referring to FIG. 3, particles from sample #1 were placed on a microscope slide and observed under the polarized light filter and a 530 nm light filter using a 40× objective. Two images are compared side by side with one image having been physically rotated on the microscope stage roughly 45° compared to the other image.

The majority of the particles exhibited birefringence under these conditions. When the particles are physically rotated on the microscope stage, their color changes. This indicates that the light was moving through two different refractive indices (RI) of the crystalline structure. Amorphous particles refract light isotropically due to the lack of order on the atomic or molecular level. This results in the particles being the same color as 530 nm light regardless of orientation. Referring to FIG. 3A) the sample #1 particles are comprised of an anisotropic crystalline structure with two different refractive indices so that when the light is passed from one RI to the other, the color changes.

Referring to FIG. 3B) the particles from the second Isowater™ protein sample show less uniform crystalline behavior. Several of the particles are the same color as the 530 nm light filter, suggesting they are amorphous (or extremely thin crystalline particles). Other particles are uniformly crystalline exhibiting comparable behavior to the particles above. Referring to FIG. 3C) a third set of particles appears to contain microcrystals that are not aligned over the dimensions of the particle. As these particles are rotated, the microcrystals changes from one color to the other suggesting that at a much smaller length scale, crystals exist that are combined into the larger particles. The majority of the particles in sample #3 are composed of the microcrystals. This suggests that at higher protein concentration, the solutions may have been saturated which resulted in seed particles for the microcrystals. At the lower protein concentration of sample #1,the solution may have become super saturated during evaporation and crystallized via a different mechanism than what is observed here. Regardless, all three Isowater™ samples appear to contain particles that are crystalline since they exhibit birefringent behavior.

Referring to FIG. 3D) the majority of the particles from the Perfect Day Whey β-lactoglobulin sample #4 exhibit crystalline behavior in polarized light. The particles here more closely resembled those in sample #1 where the particles are transparent suggesting the presence of a single crystalline phase. The presence of the microcrystalline structure within particles was not observed here.

### Example 2

### Crystalline Protein Analysis by Dynamic Vapor Sorption

Dynamic vapor sorption (DVS)—crystalline protein samples are analyzed using the ProUmid™ DVS system by drying the samples at 30 °C for up to six hours followed by exposing the samples to 10% RH to 90% RH conditions stepwise in 10% RH intervals. Changes in weight are recorded at various time intervals.

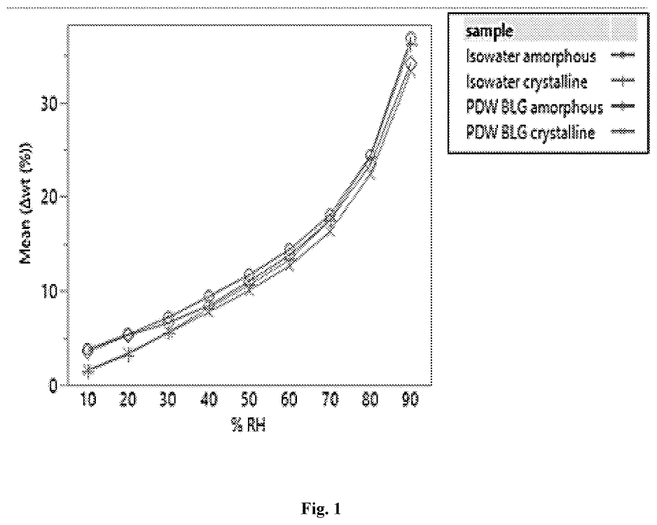

Referring to FIG. 1, the dynamic vapor sorption (DVS) results show the crystalline materials are less hygroscopic than their corresponding amorphous materials. The two replicates of the Isowater™ crystalline sample and the Perfect Day Whey β-lactoglobulin (PDW BLG) sample exhibit less water vapor weight increase values than their amorphous counterparts. This observation is consistent with the expectation that amorphous materials are more hygroscopic than crystalline materials. The results are also shown in Table 3 below showing the weight of the crystalline materials at different relative humidities compared to their corresponding amorphous material forms.

TABLE 3

| | | | 10% | 20% | 30% | 40% | 50% | 60% | 70% | 80% | 90% |
|---|---|---|---|---|---|---|---|---|---|---|---|
| Material | State | Replicate | RH | RH | RH | RH | RH | RH | RH | RH | RH |
| Isowater | Crystalline | 1 | 1.50 | 3.24 | 5.66 | 8.23 | 10.68 | 13.38 | 17.61 | 24.22 | 36.23 |
| Isowater | Crystalline | 2 | 1.52 | 3.27 | 5.62 | 8.11 | 10.55 | 13.30 | 17.52 | 24.13 | 63.12 |
| PDWBLG | Crystalline | 1 | 1.59 | 3.42 | 5.66 | 7.96 | 10.22 | 12.79 | 16.41 | 22.46 | 33.43 |
| PDWBLG | Crystalline | 2 | 1.61 | 3.38 | 5.43 | 7.58 | 9.87 | 12.54 | 46.19 | 22.25 | 33.15 |
| Isowater | Amorphous | 1 | 3.83 | 5.40 | 7.18 | 9.41 | 11.70 | 14.39 | 18.06 | 24.36 | 36.83 |
| Isowater | Amorphous | 2 | 3.82 | 5.38 | 7.16 | 9.39 | 11.68 | 14.38 | 18.05 | 24.35 | 36.77 |
| PDWBLG | Amorphous | 1 | 3.52 | 5.27 | 6.60 | 8.41 | 10.97 | 13.78 | 17.43 | 23.37 | 33.91 |
| PDWBLG | Amorphous | 2 | 3.55 | 5.30 | 6.62 | 8.45 | 11.01 | 13.83 | 17.49 | 23.48 | 34.34 |

These results indicate that it is unexpected that though the protein crystalline materials are as hygroscopic as they are, the crystalline proteins did not exhibit deliquescence after being exposed to 90% RH conditions in this study. Prior studies have shown that crystalline materials such as amino acids and organic acids exhibit virtually no water vapor sorption at lower relative humidity values, and then exhibit deliquescence above the deliquescence point (RHo), which typically occurs at relative humidity values greater than 50% RH. However, the crystalline proteins did not exhibit deliquescence after being exposed to 90% RH conditions in this study. The crystalline particles remained free flowing at the completion of this study, while the amorphous versions exhibited caking. Practically, this demonstrates the benefit of crystallizing these proteins, as this would potentially extend the shelf life of the product from a hygroscopicity standpoint.

Statistically, the amorphous phase exhibits more water vapor sorption than the crystalline phase through 70% RH at a 95% confidence level via the Tukey-Kramer analysis. This again demonstrates the benefit of the crystalline phase over the amorphous phase.

### Example 3

### Crystalline Protein Analysis by Densitometry

Nitrogen gas pycnometry (NGP)—the true density of the powders is measured using an AccuPyc II™ gas pycnometer and nitrogen gas.

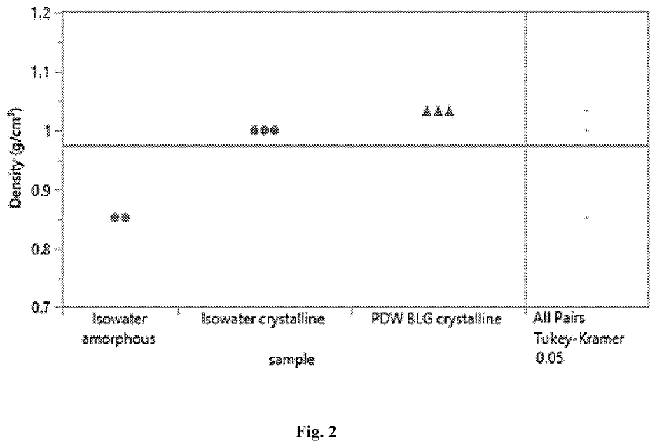

Referring to FIG. 2, the true densities of the crystalline proteins were measured using nitrogen gas and the AccuPyc II™ pycnometer, and the results show an increase in density compared to the amorphous Isowater™ protein. The crystalline Perfect Day™ Whey β-lactoglobulin protein exhibited the highest true density with a mean value of 1.03 g/cm³, followed by the crystalline Isowater™ protein at 1.00 g/cm³. The amorphous Isowater™ protein particles exhibited a true density of 0.85 g/cm³.

Since the densities of the two crystalline materials are at or greater than the highest density of pure water, the particles are expected to sink in water whereas the amorphous material will remain buoyant. This property of increased density equal to or greater than the density of water is observed when crystalline Isowater™ protein crystals are poured in water and the crystals are observed to sink rather than float on the water surface, indicating a higher density than water. This property has the potential to offer a consumer benefit of improving the dispersibility of protein powder in water if the protein is in a crystalline state.

It is expected that oral delivery of a dietary supplement, food product, or nutritional, nutraceutical, food, beverage, or supplement composition comprising a blend of crystalline protein compositions derived as described herein would be useful in supporting health in a human subject.

For example, a blend of crystalline protein, micronutrients, macronutrients and other nutritional, nutraceutical, food, beverage, and supplement composition as described above may be used to improve health of a subject.

The use of the terms “a,” “an,” “the,” and similar referents in the context of describing the present invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately +10%; in other embodiments, the values may range in value above or below the stated value in a range of approximately +5%; in other embodiments, the ° values may range in value above or below the stated value in a range of approximately +2%; in other embodiments, the values may range in value above or below the stated value in a range of approximately +1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise stated. No language in the specification should be construed as indicating any non-claimed element as essential to the practice of the invention.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entireties. The present invention may be embodied in other specific forms without departing from the spirit or essential attributes thereof, and, accordingly, reference should be made to the appended claims, rather than to the foregoing specification, as indicating the scope of the invention.

What is claimed is:

1. A dietary supplement, food product, or nutraceutical composition for use in supporting health in a human subject, comprising a crystalline protein.

2. The dietary supplement of claim 1, where the crystalline protein is combined with one or more nutritional components selected from macronutrients, micronutrients, vitamins and mineral.

3. The dietary supplement of claim 1, wherein the crystalline protein is beta lactoglobulin.

4. The dietary supplement in claim 1, wherein the crystalline protein is a collagen peptide.

5. The dietary supplement of claim 2, wherein the nutritional component is one or more macronutrient selected from carbohydrates, fats, saturated fats, monounsaturated fatty acids, polyunsaturated fatty acids, essential fatty acids, fiber, and proteins.

6. The dietary supplement of claim 3, wherein the nutritional component is one or more micronutrient selected from vitamins, minerals, choline, inositol, flavonoids, bioflavonoid, and carnitine.

7. The dietary supplement of claim 4, wherein the nutritional component is one or more vitamin selected from vitamin, vitamin A, vitamin B1, B2, B3, B5, B-6, B7 and B-12, vitamin C, vitamin D and D3, vitamin E, vitamin K, and folate.

8. The dietary supplement of claim 5, wherein the nutritional component is one or more minerals selected from boron, calcium, chromium, copper, iron, iodine, magnesium, molybdenum, potassium, selenium, zinc, and chloride.

9. The dietary supplement of claim 6, wherein the composition is administered in an oral dosage form selected from tablets, capsules, solutions, suspensions, powders, gums, and confectionaries.

10. The dietary supplement of claim 5, wherein the composition is administered in a delivery form selected from powder, sublingual dissolvable tablets dissolving under and on the tongue, and edible or dissolvable film dosage forms selected from hydrophilic polymers, oral dissolvable films, and oral dissolvable strips.

11. A method for producing a crystalline protein for use in a dietary supplement, food product, or nutraceutical formulation for use in a nutritional composition for supporting health in a human subject, comprising the step of crystallizing a protein resulting in a protein crystal having reduced hygroscopicity, improved shelf life, and low deliquescence properties.

12. The method of claim 11, wherein, the crystalline protein is whey protein.

13. The method of claim 11, further comprising the step of mixing the crystalline whey protein with one or more components selected from macro- and micro-nutritional, nutraceutical, food, and beverage composition components.

14. The method of claim 13, wherein the one or more component is one or more macro-nutritional component selected from carbohydrates, fats, saturated fats, monounsaturated fatty acids, polyunsaturated fatty acids, essential fatty acids, fiber, and proteins.

15. The method of claim 14, wherein the one or more component is one or more micro-nutritional component selected from vitamins, minerals, choline, inositol, flavonoids, bioflavonoid, and carnitine.

16. The method of claim 15, wherein the one or more component is one or more vitamin selected from vitamin, vitamin A, vitamin B1, B2, B3, B5, B-6, B7 and B-12, vitamin C, vitamin D and D3, vitamin E, vitamin K, and folate.

17. The method of claim 16, wherein the one or more component is one or more minerals selected from boron, calcium, chromium, copper, iron, iodine, magnesium, molybdenum, potassium, selenium, zinc, and chloride.

18. A method for improving shelf life and stability of a protein powder component in a dietary supplement, food, beverage, or nutraceutical formulation, comprising the step of crystallizing the protein into crystalline form of the protein having reduced hygroscopicity, higher density, dispersibility, and improved solubility properties.

19. The method of claim 18 wherein crystalline protein is whey protein.

20. The method of claim 19 further comprising the step of mixing the crystalline whey protein with one or more components selected from macro- and micro-nutritional, nutraceutical, food, and beverage composition components; wherein the one or more component is one or more macro-nutritional component selected from carbohydrates, fats, saturated fats, monounsaturated fatty acids, polyunsaturated fatty acids, essential fatty acids, fiber, and proteins; wherein the one or more component is one or more micro-nutritional component selected from vitamins, minerals, choline, inositol, flavonoids, bioflavonoid, and carnitine; wherein the one or more component is one or more vitamin selected from vitamin, vitamin A, vitamin B1, B2, B3, B5, B-6, B7 and B-12, vitamin C, vitamin D and D3, vitamin E, vitamin K, and folate, and is one or more minerals selected from boron, calcium, chromium, copper, iron, iodine, magnesium, molybdenum, potassium, selenium, zinc, and chloride.
